(19) United States
(12) Patent Application Publication
(10) Pub. No.: US 2001/0034330 A1
(43) Pub. Date: Oct. 25, 2001

(54) Innate immunity-stimulating compositions of CpG and saponin and methods thereof

(76) Inventor: Charlotte Kensil, Milford, MA (US)

Correspondence Address: Colleen Superko, Hale and Dorr LLP, 60 State Street, Boston, MA, 02109, US

(21) Appl. No.: 09/760,506

(22) Filed: Jan. 12, 2001

Related U.S. Application Data
(60) Provisional application No. 60/200,853, filed on May 1, 2000.
(60) Provisional application No. 60/175,840, filed on Jan. 13, 2000.
(60) Provisional application No. 60/128,608, filed on Apr. 8, 1999.
(60) Provisional application No. 60/095,913, filed on Aug. 10, 1998.

(51) Int. Cl. A61K 48/00

(52) U.S. Cl. 514/44; 514/26; 514/33

(57) ABSTRACT

Compositions comprising oligonucleotides comprising at least one unmethylated CpG dinucleotide and saponin and the use thereof for stimulating innate immunity and enhancing natural killer cell activity are disclosed.

### CROSS-REFERENCE TO RELATED APPLICATION

This U.S. Utility Patent Application claims priority from U.S. Provisional Application No. 60/200,853, filed May. 1, 2000, U.S. Provisional Application No. 60/175,840, filed Jan. 13, 2000 and U.S. Utility patent application Ser. No. 09/369,941, filed Aug. 6, 1999, which claims benefit of U.S. Provisional Application No. 60/128,608, filed Apr. 8, 1999, now abandoned, and U.S. Provisional Application No. 60/095,913, filed Aug. 10, 1998, now abandoned.

### FIELD OF THE INVENTION

The present invention is in the field of immune enhancers. The compositions of the invention stimulate innate immunity.

### BACKGROUND OF THE INVENTION

Adjuvant saponins have been identified and purified from an aqueous extract of the bark of the South American tree, Quillaja saponaria Molina. Among the 22 saponin peaks which were separable, the more predominant purified saponins have been identified as QS-7, QS-17, QS-18, and QS-21, also known as QA-7, QA-17, QA-18, and QA-21, respectively. These saponins have been substantially purified by various methods including high pressure liquid chromatography (“HPLC”), low pressure liquid silica chromatography, and hydrophilic interactive chromatography (“HILIC”). The substantially pure saponins have been found to be useful as immune adjuvants when used with vaccine antigens for enhancing immune responses to such antigens in individuals. (Kensil, et al., U.S. Pat. No. 5,057,540; Kensil, et al., J. Immunol. 148:2357 (1991); Marciani, et al., Vaccine 9:89 (1991).)

Recently, oligonucleotides containing the unmethylated cytosine-guanine (“CpG”) dinucleotide in a particular sequence context or motif have been shown to be potent stimulators of several types of immune cells in vitro. (Weiner, et al., Proc. Natl. Acad. Sci. 94:10833 (1997).) An oligonucleotide comprising an unmethylated CpG motif within which is at least one unmethylated CpG dinucleotide has been shown to activate the immune system. CpG motifs can stimulate monocytes, macrophages, and dendritic cells that can produce several cytokines, including the T helper 1 (“Th 1”) cytokine interleukin (“IL”) 12. (Carson, et al., J. Exp. Med. 186:1621 (1997).) This effect causes the induction of IFN-γ secretion by natural killer (NK) cells, which in turn, activates macrophages and enhances immunoglobulin isotype switching to IgG2a, a hallmark of T helper Type 1 cell immunity and differentiation. (Chu, et al., J. Exp. Med. 186:1623 (1997).) Klinman, et al., have shown that a DNA motif consisting of an unmethylated CpG dinucleotide flanked by two 5′ purines (GpA or ApA) and two 3′ pyrimidines (TpC or TpT) optimally stimulated B cells to produce IL-6 and IL-12, and stimulated CD4+ T cells to produce IL-6 and IFN-γ both in vitro and in vivo. (Klinman, et al., Proc. Natl. Acad. Sci., 93:2879 (1996).) Davis, et al., discovered that nucleic acids containing at least one unmethylated CpG dinucleotide may affect the immune response of a subject (Davis, et al., WO 98/40100). Kensil, et al., previously showed that a combination of QS-21 and CpG oligonucleotides have synergistic adjuvant activity for antigen-specific responses when combined with a vaccine antigen (Kensil, U.S. Ser. No. 09/369,941, the contents of which are fully incorporated by reference herein).

Recently, it has been shown that CpG administration, in the absence of a vaccine antigen, can protect a mouse against an otherwise lethal infection with an intracellular bacteria, such as Listeria monocytogenes or Francisella tularensis, if the CpG is administered between 2-3 days prior or no earlier than 2 weeks prior to the infection. (Elkins, et al., J. Immunol. 162: 2991 (1999).). This result suggests an activation of innate immunity. It has been hypothesized that CpG motifs are a danger signal that activate protective innate immune defenses (Krieg, et al., J. Immunol. 161: 2428 (1998)), in particular (NK) cell activity. CpG motifs appear to stimulate natural killer cell activity through direct CpG stimulation of natural killer cells or through natural killer-active cytokines secreted by CpG-stimulated monocytes.

### SUMMARY OF THE INVENTION

Since innate immunity plays an important role in the protective response to infection with certain microbial agents and tumors, a need exists to characterize other agents that may safely stimulate innate immunity. Such agents may be potentially incorporated in future therapeutic agents. Surprisingly, a combination of an oligonucleotide comprising at least one unmethylated CpG dinucleotide and a saponin was found to be a powerful stimulator of natural killer cell activity compared to either compound alone. NK cell activity was significantly higher for a composition comprising a CpG-containing oligonucleotide/saponin combination compared to either the saponin or the unmethylated CpG-containing oligonucleotide and represented a positive synergistic effect. Further, the saponin alone was shown to induce a higher natural killer cell response than the unmethylated CpG-containing oligonucleotide. Further, both the saponin alone and the combination of saponin/a CpG-containing oligonucleotide induced an innate immunity that enabled stronger protection against an infection than the CpG-containing oligonucleotide. Together, these results establish that a composition comprising saponin alone and a composition comprising an oligonucleotide comprising at least one unmethylated CpG dinucleotide plus a saponin are candidate compositions to induce innate immunity.

Accordingly, in a first aspect, the invention covers a composition comprising: (a) a saponin; and (b) an oligonucleotide comprising at least one unmethylated CpG dinucleotide. Preferably, the composition provides that the saponin is derived from Quillaja saponaria, and more preferably, the saponin is chemically modified or comprises a substantially pure saponin. In a preferred embodiment of the first aspect, the substantially pure saponin comprises QS-7, QS-17, QS-18, or QS-21, and more preferably, the substantially pure saponin comprises QS-21. In yet other preferred embodiments of the first aspect, the composition is further directed to one in which the oligonucleotide is chemically modified. More particularly, the oligonucleotide is modified with at least one phosphorothioate internucleotide linkage. A preferred embodiment of the first aspect encompasses the composition wherein the oligonucleotide comprises a CpG motif having the formula 5′X₁CGX₂3′, wherein at least one nucleotide separates consecutive CpGs, and wherein X₁, is adenine, guanine, or thymineand X₂ is cytosine, thymine, or adenine. More preferably, the CpG motif comprises TCTCCCAGCGTGCGCCAT or TCCATGACGTTCCTGACGTT or TCGTCGTTTTGTCGTTTTGTCGTT. The composition, according to the first aspect of the invention, preferably increases an innate immune response when administered to a mammal or a human. Still another preferred embodiment is directed to the composition wherein the composition enhances a natural killer cell response, preferably in a positive synergistic manner.

In a second aspect, the invention is directed to a method for stimulating innate immunity comprising administering an effective amount of a composition comprising: (a) a saponin; and (b) an oligonucleotide comprising at least one unmethylated CpG motif to an individual. Preferably, the method provides that the saponin is derived from Quillaja saponaria, and more preferably, the saponin is chemically modified or comprises a substantially pure saponin. In a preferred embodiment of the second aspect, the substantially pure saponin comprises QS-7, QS-17, QS-18, or QS-21, and more preferably, the substantially pure saponin comprises QS-21. In yet other preferred embodiments of the second aspect, the method is further directed to one in which the oligonucleotide is chemically modified. More particularly, the oligonucleotide is modified with at least one phosphorothioate internucleotide linkage. A preferred embodiment of the second aspect encompasses the method wherein the oligonucleotide comprises a CpG motif having the formula 5′X₁CGX₂3′, wherein at least one nucleotide separates consecutive CpGs, and wherein X₁ is adenine, guanine, or thymine, and X₂ is cytosine, thymine, or adenine. More preferably, the CpG motif comprises TCTCCCAGCGTGCGCCAT or TCCATGACGTTCCTGACGTT or TCGTCGTTTTGTCGTTTTGTCGTT. The method, according to this second aspect of the invention, preferably further increases an innate immune response when administered to a mammal or a human. Still another preferred embodiment is directed to the method for further enhancing a natural killer cell response, preferably in a positive synergistic manner.

A third aspect of the invention provides for methods for stimulating innate immunity comprising administering an effective amount of a composition comprising a saponin only to an individual. Preferably, the method provides that the saponin is derived from Quillaja saponaria, and more preferably, the saponin is chemically modified or comprises a substantially pure saponin. In a preferred embodiment of the third aspect, the substantially pure saponin comprises QS-7, QS-17, QS-18, or QS-21, and more preferably, the substantially pure saponin comprises QS-21. The method, according to the third aspect of the invention, preferably further increases an innate immune response when administered to a mammal or a human. Still another preferred embodiment is directed to the method for further enhancing a natural killer cell response.

### DESCRIPTION OF THE FIGURES

FIG. 1 is a graphic representation showing the enhancement of the natural killer cell response by QS-21 or by QS-21/CpG oligodeoxynucleotide (sequence 1826) combination, as evidenced by lysis of the NK-sensitive cell line YAC-1.

FIG. 2 is a graphic representation showing the optimal timing of administration of QS-21/CpG oligodeoxynucleotide (sequence 1826) combination, as evidenced by lysis of the NK-sensitive cell line YAC-1.

FIG. 3 is a graphic representation depicting the NK activating activity of QS-21, QS-7, or CpG oligodeoxynucleotide (sequence 1826), as evidenced by dose response curves for individual compounds for enhancing the NK cell response against the NK-sensitive cell line YAC-1.

FIG. 4 is a graphic representation depicting the NK activating activity of various mixtures of QS-21, QS-7, and CpG oligodeoxynucleotides (sequences 1826 and 2006), as evidenced by lysis of the NK-sensitive cell line YAC-1.

FIG. 5 is a graphic representation illustrating protection of Balb/c mice against an intraperitoneal challenge with 10⁵ colonies of Listeria monocytogenes after administration of various formulations three days prior to challenge.

### DESCRIPTION OF THE PREFERRED EMBODIMENTS

The term “saponin” as used herein includes glycosidic triterpenoid compounds which produce foam in aqueous solution and have hemolytic activity in most cases. The invention encompasses the saponin per se, as well as natural and pharmaceutically acceptable salts and pharmaceutically acceptable derivatives. The term “saponin” also embodies biologically active fragments thereof. The term “saponin” also encompasses chemically modified saponins.

The saponins of the present invention may be obtained from the tree Quillaja saponaria Molina. (Dalsgaard, Acta Veterinia Scandinavica, 69:1 (1978).) A partially purified saponin enriched extract, prepared as described by Dalsgaard, (“Quil-A”) has adjuvant activity. Such an extract can be further separated. Among the 22 saponin peaks which were separable, the more predominant purified saponins have been identified as QS-7, QS-17, QS-18, and QS-21, also known as QA-7, QA-17, QA-18, and QA-21, respectively. (Kensil, et al., U.S. Pat. No. 5,057,540.) These saponins have been substantially purified by various methods including HPLC, low pressure liquid silica chromatography, and HILIC.

“QS-21” designates the mixture of components QS-21-V1 and QS-21-V2 which appear as a single peak on reverse phase HPLC on Vydac C4 (5 μm particle size, 300A pore, 4.6 mm ID×25 cm length) in 40 mM acetic acid in methanol/water (58/42, v/v). The component fractions are referred to specifically as QS-21-V1 and QS-21-V2 when describing experiments performed on the further purified components.

The present invention may also employ chemically modified saponins. According to Kensil, et al., U.S. Pat. No. 5,443,829, the contents of which are fully incorporated by reference herein, such chemically modified saponins can be obtained in several ways. For example, the aldehyde group of either purified QS-17, QS-18, QS-21, or mixtures thereof, or purified fractions obtainable from Quillaja saponaria Molina bark and comprising QS-17, QS-18, and QS-21 can be reduced with a mild reducing agent, such as sodium or lithium borohydride, to give the corresponding alcohol. Alternatively, the aldehyde of QS-17, QS-18, and QS-21, mixtures thereof, or purified fractions obtainable from Quillaja saponaria Molina bark and comprising QS-17, QS-18, and QS-21 can be subjected to reductive amidation with a primary amine and a reducing agent to give the corresponding amino derivative of QS-17, QS-18, and QS-21. According to Kensil, et al., U.S. Pat. No. 5,583,112, the contents of which are fully incorporated by reference herein, the carboxyl group on the glucuronic acid of saponins from Quillaja saponaria Molina can be conjugated to a protein, a peptide, or a small molecule containing a primary amine. According to Higuchi, et al., Phytochemistry 26:229 (1987)), saponins from Quillaja saponaria may be deacylated by alkaline-catalyzed hydrolysis. According to Marciani, et al., U.S. Pat. No. 5,977,081, the contents of which are fully incorporated by reference herein, the carboxyl group on the glucuronic acid of nonacylated or deacylated saponins from Quillaja saponaria may be conjugated to a lipid, fatty acid, polyethylene glycol, or terpene. Thus, the present invention relates to a chemically modified saponin or a biologically active fraction thereof obtainable from a crude Quillaja saponaria Molina extract. Adjuvant-active saponins and adjuvant-inactive saponins fall within the scope of the invention described herein provided that these saponins stimulate innate immunity alone or in combination with a CpG dinucleotide.

In other embodiments of the invention, the term “saponin” covers mixtures of saponins. Preferably, the mixture of saponins comprises two or more substantially pure saponins. More preferably, the two or more substantially pure saponins are from Quillaja saponaria in doses that are otherwise suboptimal for the individual saponins. In a particularly preferred embodiment, the combination of saponins consists essentially of substantially pure saponins QS-7and QS-21 or, in other particularly preferred embodiments, QS-7 and QS-21-V1 or QS-7 and QS-21-V2.

Other embodiments of the invention encompasses saponins in combination with excipients. Preferably, the saponin is QS-21 and the excipients are selected from nonionic surfactants, polyvinyl pyrolidone, human serum albumin, aluminum hydroxide, agents with anesthetic action, and various unmodified and derivatized cyclodextrins. More preferably, in these embodiments, the nonionic surfactants are selected from Polysorbate 20, Polysorbate-40, Polysorbate-60, and Polysorbate-80. The polyvinyl pyrolidone may preferably be Plasdone C15, a pharmaceutical grade of polyvinyl pyrolidone. The agent having anesthetic action preferably is benzyl alcohol. Preferred cyclodextrins are hydroxypropyl-β-cyclodextrin, hydroxypropyl-γcycodextrin, methyl-β-cyclodextrin.

The term “partially pure” means saponins partially separated from compounds normally associated with the saponin in its natural state.

The term “substantially pure” means substantially free from compounds normally associated with the saponin in its natural state and exhibiting constant and reproducible chromatographic response, elution profiles, and biologic activity. The term “substantially pure” is not meant to exclude artificial or synthetic mixtures of the saponin with other compounds.

The present invention may also employ saponins isolated from other plant species, such as Gypsophila or Saponaria officinalis.

In one embodiment, the invention provides a method for stimulating an immune response in a subject by administering a therapeutically effective amount of saponin and oligonucleotide comprising at least one unmethylated CpG dinucleotide. The term “nucleic acid” or “oligonucleotide” refers to a polymeric form of nucleotides at least five bases in length. The nucleotides of the invention can be deoxyribonucleotides, ribonucleotides, or modified forms of either nucleotide. Generally, double-stranded molecules are more stable in vivo, while single-stranded molecules have increased activity.

The nucleic acid molecule can include the use of phosphorothioate or phosphorodithioate rather than phosphodiesterase linkages within the backbone of the molecule, or methlyphosphorothioate terminal linkages (Kriege, et al., Antisense and Nucl Acid Drug Dev 6:133 (1996); Bosggs, et al., Antisense and Nucl Acid Drug Dev, 7:461 (1997). The phosphate backbone modification can occur at the 5′end of the nucleic acid. The phosphate backbone modification m ay occur at the 3′end of the nucleic acid, for example at the last five nucleotides of the 3′end of the nucleic acid. Hutcherson, et al., reports in WO 95/26204 the nonsequence-specific immunostimulatory effect of phosphorothioate modified oligonucleotides. Nontraditional bases such as inosine and queosine, as well as acetyl-,thio-and similarly modified forms of adenine, cytidine, guanine, thymine, and uridine can also be included, which are not as easily recognized by endogenous endonucleases. Other stabilized nucleic acid molecules include: nonionic DNA analogs, such as alkyl-and aryl-phosphonates (in which the charged oxygen moiety is alkylated). Nucleic acid molecules which contain a diol, such as tetrahyleneglycol or hexaethleneglycol, at either or both termini are also included. The term “oligonucleotide” includes both single and double stranded forms of DNA.

A “CpG” or “CpG motif” refers to a nucleic acid having a cytosine followed by a guanine linked by a phosphate bond. The term “methylated CpG” refers to the methylation of the cytosine on the pyrimidine ring, usually occurring the 5-position of the pyrimidine ring. The term “unmethylated CpG” refers to the absence of methylation of the cytosine on the pyrimidine ring. Methylation, partial removal, or removal of an unmethylated CpG motif in an oligonucleotide of the invention is believed to reduce its effect. Methylation or removal of all unmethylated CpG motifs in an oligonucleotide substantially reduces its effect. The effect of methylation or removal of a CpG motif is “substantial” if the effect is similar to that of an oligonucleotide that does not contain a CpG motif. In a preferred embodiment, the CpG motif is an unmethylated CpG dinucleotide.

Preferably the CpG oligonucleotide is in the range of about 5 to 40 bases in size. For use in the instant invention, the nucleic acids can be synthesized de novo using any of a number of procedures well known in the art. For example, the b-cyanoethyl phosphoramidite method (Beaucage, et al., Tet. Let. 22: 1859 (1981); nucleoside H-phosphonate method (Garegg, et al., Tet. Let. 27: 4051, (1986); Froehler, et al., Nucl. Acid. Res. 14:5399 (1986); Garegg, et al., Tet. Let. 27:4055 (1986); and Gaffney, et al., Tet. Let. 29:2619 (1998)). These chemistries can be performed by a variety of automated oligonucleotide synthesizers available in the market. Alternatively, CpG dinucleotides can be produced on a large scale in plasmids, (see Sambrook, T., et al., Molecular Coning: A Laboratory Manual, Cold Spring Harbor Laboratory Press, New York, 1989) which after being administered to a subject are degraded into oligonucleotides. Oligonucleotides can be prepared from existing nucleic acid sequences (e.g., genomic or cDNA) using known techniques, such as those employing restriction enzymes, exonucleases or endocucleases.

For use in vivo, nucleic acids are preferably relatively resistant to degradation (e.g., via endo- and exo-nucleases). Secondary structures, such as stem loops, can stabilize nucleic acids against degradation. Alternatively, nucleic acid stabilization can be accomplished via phosphate backbone modifications. A preferred stabilized nucleic acid has at least a partial phosphorothiate modified backbone. Phosphorothioates may be synthesized using automated techniques employing either phosphoramidate or H-phosphonate chemistries. Aryl-and alkyl-phosphonates can be made, e.g., as described in Ts'O , et al., U.S. Pat. No. 4,469,863; and alkylphosphotriesters (in which the charged oxygen moiety is alkylated as described in Tullis, U.S. Pat. No. 5,023,243 and Tullis, EP 092574B1) can be prepared by automated solid phase synthesis using commercially available reagents. Methods for making other DNA backbone modifications and substitutions have been described (Uhlmann, et al., Chem. Rev. 90: 544 (1990); Goodchild, Bioconjugate Chem. 1:165 (1990).

For administration in vivo, nucleic acids may be associated with a molecule that results in higher affinity binding to target cell (e.g., B-cell, monocytic cell and natural killer (NK) cell) surfaces and/or increased cellular uptake by target cells to form a “nucleic acid delivery complex.” Nucleic acids can be ionically or covalently associated with appropriate molecules using techniques which are well known in the art. A variety of coupling or cross-linking agents can be used, e.g., protein A, carbodiimide, and N-succinimidyl-3-(2-pyridyldithio) propionate (SPDP). Nucleic acids can alternatively be encapsulated in liposomes or virosomes using well-known techniques.

In preferred embodiments, the oligonucleotide containing the CpG motif may be part of a monomer or part of a multimer. Alternatively, the CpG motif may be a part of the sequence of a vector.

One embodiment of the invention covers the oligonucleotide which contains a CpG motif having the formula 5′X₁,CGX₂3′, wherein at least one nucleotide separates consecutive CpGs, and wherein X₁ is adenine, guanine, or thymine, and X₂ is cytosine, thymine, or adenine.

In another embodiment, the oligonucleotide sequences useful in the methods of the invention are represented by the formula:

5′N₁,XCGX₂N₂3′

wherein at least one nucleotide separates consecutive CpGs; X₁ is adenine, guanine, or thymidine; X₂ is cytosine or thymine, N is any nucleotide and N₁ +N₂ is from about 0.26 bases. In a preferred embodiment, N₁ and N₂ do not contain a CCGG quadmer or more than one CGG trimer; and the nucleic acid sequence is from about 8-30 bases in length. However, nucleic acids of any size (even may kb long) can be used in the invention if CpGs are present, as larger nucleic acids are degraded into oligonucleotides inside cells. Preferred synthetic oligonucleotides do not include a CCGG quadmer or more than one CCG or CGG trimer at or near the 5′ or 3′ terminals and/or the consensus mitogenic CpG motif is not a palindrome. A “palindromic sequence” or “palindrome” means an inverted repeat (i.e., a sequence such as ABCDEE'D'C'B'A', in which A and A' are bases capable of forming the usual Watson-Crick base pairs.

In still another embodiment, the method of the invention includes the use of an oligonucleotide which contains a CpG motif represented by the formula:

5′N₁X₁X₂CGX₃X₄N₂3′

wherein at least one nucleotide separates consecutive CpGs; X₁X₂ is selected from the group consisting of GpT, GpG, GpA, ApT and ApA; X₃X₄ is selected from the group consisting of TpT or CpT; N is any nucleotide and N₁,+N₂ is from about 0-26 bases. In a preferred embodiment, N₁, and N₂ do not contain a CCGG quadmer or more than one CCG or CGG trimer. CpG oligodeoxynucleotides are also preferably in the range of 8 to 30 bases in length, but may be of any size (even many kb long) if sufficient motifs are present, since such larger nucleic acids are degraded into oligonucleotides inside of cells. Preferred synthetic oligonucleotides of this formula do not include a CCGG quadmer or more than one CCG or CGG trimer at or near the 5′ and/or 3′ terminals and/or the consensus mitogenic CpG motif is not a palindrome. Other CpG oligonucleotides can be assayed for efficacy using methods described herein.

In a preferred embodiment, the CpG motif comprises TCTCCCAGCGTGCGCCAT (also known as “CpG sequence 1758”) or TCCATGACGTTCCTGACGTT (also known as “CpG sequence 1826”) or TCGTCGTTTTGTCGTTTTGTCGTT (also known as “CpG sequence 2006”).

The oligonucleotides of the invention may be chemically modified in a number of ways in order to stabilize the oligonucleotide against endogenous endonucleases. According to Davis, et al., WO 98/40100, a prolonged effect can be obtained using stabilized oligonucleotides, where the oligonucleotide incorporates a phosphate backbone modification (e.g., a phosphorothioate or phosphorodithioate modification). For example, the oligonucleotides may contain other than phosphodiester internucleotide linkages between the 5′ end of one nucleotide and the 3′ end of another nucleotide in which the other linkage, the 5′ nucleotide phosphate, has been replaced with any number of non-traditional bases or chemical groups, such as phosphorothioate. More particularly, the phosphate backbone modification occurs at the 5′ end of the nucleic acid for example, at the first two nucleotides of the 5′ end of the nucleic acid. Further, the phosphate backbone modification may occur at the 3′ end of the nucleic acid for example, at the last five nucleotides of the 3′ end of the nucleic acid. The oligonucleotide comprising at least one unmethylated CpG dinucleotide may preferably be modified with at least one such phosphorothioate internucleotide linkages.

Oligonucleotides with phosphorothioate linkages may be prepared using methods well known in the field such as phosphoamidite (Agrawal, et al., Proc. Natl. Acad. Sci. 85:7079 (1988)) or H-phosphonate (Froehler, et al., Tetrahedron Lett. 27:5575 (1986)). Examples of other modifying chemical groups include alkylphosphonates, phosphorodithioates, alkylphosphorothioates, phosphoroamidates, 2-0-methyls, carbamates, acetamidates, carboxymethylesters, carbonates, and phosphate triesters. Oligonucleotides with these linkages can be prepared according to know methods (Goodchild, Chem. Rev. 90:543 (1990); Uhlmann, et al., Chem. Rev. 90:534 (1990); and Agrawal, et al., Trends Biotechnol. 10:152 (1992)).

In a preferred embodiment of this aspect, the inventive compositions activate the immune system. Certain preferred nucleic acids containing an unmethylated CpG have a relatively high stimulation with regard to B cell, monocyte, and/or NK cell responses. For example, as assayed by induction of cytokines, proliferative responses, lytic responses, the stimulation of the immune system may be determined.

Nucleic acids containing an unmethylated CpG can be effective in any mammal, preferably a human. Different nucleic acids containing an unmethylated CpG can cause optimal immune stimulation depending on the mammalian species. Thus, an oligonucleotide causing optimal stimulation in humans may not cause optimal stimulation in a mouse. One of skill in the art can identify the optimal oligonucleotides useful for a particular mammalian species of interest.

The term “innate immunity” as used herein refers to an immune response that is independent of a specific vaccine antigen. Cellular components involved in innate immune responses include monocytes, macrophages, natural killer cells, and polymorphonuclear cells, such as neutrophils. The term “nonspecific immunostimulator” refers to compounds that when administered to an individual or tested in vitro, increase the innate immunity of that individual or test system. Preferably, such individuals are mammals, and more preferably, the mammals are humans, however, the invention is not intended to be so limiting. Any animal which may experience the beneficial effects of the compositions of the invention are within the scope of animals which may be treated according to the claimed invention. A nonspecific immunostimulator may enhance the immune response of the individual by increasing natural killer cell activity or cytokine production, such as interleukin-12 (IL-12) or IFNγ.

The ability of a composition to enhance innate immunity may be determined by a number of methods known to those of ordinary skill in the art. For example, the increase in natural killer cell response in mice after administration of a composition may be used as a criterion for stimulation of innate immunity. Briefly, one such method involves injecting Balb/c mice at days 1 and 2 with a test composition. Splenocytes harvested from the mice on day 3 can be tested for a natural killer cell lytic activity against a natural killer cell sensitive-cell line, such as YAC-1 cells. An additional method of determining innate immunity is to administer a test composition to a suitable species such as Balb/c mice. These mice can be challenged with an infectious agent, e.g., a bacterium such as Listeria monocytogenes after the administration of the test compound. The ability of the test compound to stimulate the innate immune response can be tested, for example, by measuring protection against infection with the infectious agent. For example, as described herein, three days after the challenge with Listeria, the spleens can be removed and tested for colony forming units of Listeria per gram as a measure of the protective benefit of the composition.

In a first aspect of the invention, a composition comprising a saponin and an oligonucleotide comprising at least one unmethylated CpG dinucleotide may be administered. More preferably, such a composition may increase the innate immune response in an individual or a test system to which the composition is administered. Preferably, the saponin is a saponin from Quillaja saponaria Molina. More preferably, the saponin is a partially pure or substantially pure saponin from Quillaja saponaria Molina. Preferably, the partially pure saponin may comprise QS-7, QS-17, QS-18, and/or QS-21 and may comprise other saponins. Preferably, the substantially pure saponin is QS-7, QS-17, QS-18, or QS-21. Most preferably, the substantially pure saponin is QS-21. Alternatively, the composition may comprise more than one saponin with the oligonucleotide comprising at least one unmethylated CpG dinucleotide.

In a further preferred embodiment of this first aspect, the saponin may cover a chemically modified saponin or a biologically active fraction thereof obtainable from a crude Quillaja saponaria Molina extract, wherein the chemically modified saponin or biologically active fraction thereof comprises at least one of QS-17, QS-18, QS-21, QS-21-V1, and QS-21-V2. The oligonucleotide comprising at least one unmethylated CpG dinucleotide is preferably a monomer or multimer. Another preferred embodiment of the CpG motif is as a part of the sequence of a vector.

Yet another embodiment of this first aspect is directed to the oligonucleotide comprising at least one unmethylated CpG dinucleotide, wherein the oligonucleotide is modified. The particular modification may comprise at least one phosphorothioate internucleotide linkage. Further, the oligonucleotide having at least one unmethylated CpG dinucleotide may comprise a CpG motif having the formula 5′X₁,CGX₂3′, wherein at least one nucleotide separates consecutive CpGs, and wherein X₁, is adenine, guanine, or thymine, and X₂ is cytosine, thymine, or adenine. The CpG motif may preferentially be TCTCCCAGCGTGCGCCAT or TCCATGACGTTCCTGACGTT, or TCGTCGTTTTGTCGTTTTGTCGTT.

The term “composition” herein refers to a composition capable of stimulating an innate immune response. A composition, according to the invention, would produce innate immunity against disease in individuals. A composition comprising a saponin and an oligonucleotide comprising at least one unmethylated CpG dinucleotide of the present invention may be administered to an individual to enhance the immune response prior to or after exposure to a pathogen or tumor. Preferably, the composition stimulates innate immunity. More preferably, the composition enhances a protective natural killer cell response.

The composition of the invention comprising both saponin and CpG-containing oligonucleotide may enhance the immune response, e.g., the innate immune response, in a positive synergistic manner. In one embodiment, the innate immune response is natural killer cell response. The term “positive synergistic effect” and “positive synergistic manner” mean the enhancement by the inventive composition, e.g., a saponin plus a CpG-containing oligonucleotide, on immune response to a level that is greater than the addition of the response to the components used individually. The synergistic effect of the composition of oligonucleotide plus saponin described herein may be shown as an increase in the maximum expected immune response, e.g, the NK cell response, over the addition of the response caused by the oligonucleotide alone and the response caused by the saponin alone.

In a second aspect, the invention is directed to a method for increasing the innate immune response in an individual or a test system comprising administering an effective amount of a composition comprising a saponin with or without an oligonucleotide comprising at least one unmethylated CpG dinucleotide. Preferably, the saponin is a saponin from Quillaja saponaria Molina. More preferably, the saponin is a partially pure or a substantially pure saponin from Quillaja saponaria Molina. The method may also embody a composition comprising more than one substantially pure saponin and an oligonucleotide comprising at least one unmethylated CpG dinucleotide. The substantially pure saponin is preferably QS-7, QS-17, QS-18, or QS-21.Most preferably, the substantially pure saponin is QS-21. In a further preferred embodiment, the saponin may cover a chemically modified saponin or a biologically active fraction thereof obtainable from a crude Quillaja saponaria Molina extract. In a preferred embodiment of the method, the oligonucleotide containing at least one CpG motif is preferably a monomer or a multimer. Another preferred embodiment of the method includes the CpG motif as a part of the sequence of a vector. Yet another embodiment is directed to the method wherein the oligonucleotide comprises at least one unmethylated CpG dinucleotide, and wherein furthermore the oligonucleotide may be chemically modified to stabilize the oligonucleotide against endogenous endonucleases. The modification may comprise at least one phosphorothioate internucleotide linkage. Further, the method may be directed, in part, to the oligonucleotide having at least one unmethylated CpG dinucleotide comprising a CpG motif having the formula 5′X₁,CGX₂3′, wherein at least one nucleotide separates consecutive CpGs, and wherein X₁ is adenine, guanine, or thymine, and X₂ is cytosine, thymine, or adenine. In another preferred method, the unmethylated CpG motif is TCTCCCAGCGTGCGCCAT, TCCATGACGTTCCTGACGTT, or TCGTCGTTTTGTCGTTTTGTCGTT.

A third aspect of the invention provides for methods for stimulating innate immunity comprising administering an effective amount of a composition comprising a saponin to an individual. Preferably, the method provides that the saponin is derived from Quillaja saponaria, and more preferably, the saponin is chemically modified or comprises a substantially pure saponin. In a preferred embodiment of the third aspect, the substantially pure saponin comprises QS-7, QS-17, QS-18, or QS-21, and more preferably, the substantially pure saponin comprises QS-21. The method, according to the third aspect of the invention, preferably further increases an innate immune response when administered to a mammal or a human. Still another preferred embodiment is directed to the method for further enhancing a natural killer cell response.

Further, numerous infectious diseases and cancers are suitable for prevention or treatment by the enhanced innate immune response. Viral diseases that can be treated or prevented by the methods of the present invention include, but are not limited to, those caused by hepatitis type A, hepatitis type B, hepatitis type C, feline leukemia virus, feline immunodeficiency virus, influenza, varicella, adenovirus, herpes simplex type I (HSV-I), herpes simplex type II (HSV-II), rinderpest, vhinovirus, echovirus, rotavirus, respiratory syncytial virus, papilloma virus, papova virus, cytomegalovirus, echinovirus arbovirus, huntavirus, coxsachie virus, mumps virus, measles virus, rubella virus, polio virus, human immunodeficiency virus type I (HIV-I), human immunodeficiency virus type II (HIV-II), rabies virus, and hoof and mouth virus.

Bacterial diseases than can be treated or prevented by methods of the present inventions are caused by bacteria including, but not limited to, mycobacteria rickettsia, mycoplasma, neisseria, legionella, Yersinia, Helobacter pylori, Staphylococcus aureus, anthrax, diphtheria, Escherichia coli, Lyme disease, Listeria monocytogenes,pneumococcus, Francisella tularensis, Salmonella, or tuberculosis.

Protozoal diseases that can be treated or prevented by the methods of the present invention are caused by protozoa including, but not limited to, leishmania, kokzidioa, trypanosoma, Plasmodium and Babeosis bovis.

Parasitic diseases that can be treated or prevented by the methods of the present invention are caused by parasites including, but not limited to, chlamydia and rickettsia. Other pathogens not listed above may be suitable for treatment by the enhanced innate immune response. In addition, cancers may be suitable for treatment by the enhanced innate immune response. Cancers that can be treated or prevented by the methods of the present invention include, but not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non Hodgkin's disease), multiple myeloma, Waldenstrobm's macroglobulinemia, and heavy chain disease.

In a specific embodiment the cancer is metastatic. In another specific embodiment, the patient having a cancer is immunosuppressed by reason of having undergone anticancer therapy (e.g., chemotherapy radiation) prior to administration of the compositions of the invention. In another specific embodiment, the cancer is a tumor.

The saponins and oligonucleotides comprising at least one unmethylated CpG dinucleotide (also referred to herein as “active compounds”) of the invention can be incorporated into pharmaceutical compositions suitable for administration. Such compositions typically comprise a saponin and an oligonucleotide comprising at least one unmethylated CpG dinucleotide and a pharmaceutically acceptable carrier. As used herein the language “pharmaceutically acceptable carrier” is intended to include any and all solvents, dispersion media, coatings, antibacterial and antifungal agent, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrocholoric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringbility exists. It must be stable under the conditions of manufacture and storage and must be preserved against the contaminating action of microorganisms such as bacteria and fungi. The carrier also can be a solvent or dispersion medium containing, for example, sterile water, salt solutions (such as Ringer's solution or saline), alcohols, gelatin, talc, viscous paraffin, fatty acid asters, hydroxymethylcellulose, polyvinyl pyrolidone, calcium carbonate, carbohydrates such as lactose, sucrose, dextrose, mannose, albumin, starch, cellulose, silica gel, polyethylene glycol (PEG), dried skim milk, rice flour, magnesium stearate, and the like, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, paragens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g. with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polyactic acid.

Methods for preparation of such formulations will be apparent to those skilled in the art. The materials can also be obtained commercially from Alza Corporation and Nova Pharmaceuticals, Inc.. Liposomal suspensions (including liposomes targeted to infected cells with monoclonal antibodies to viral antigens) can also be used as pharmaceutically acceptable carriers. These can be prepared according to methods known to those skilled in the art, for example, as described in Eppstein, et al., U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitation inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from assays and animal models described herein. Such information can be used to more accurately determine useful doses in humans.

The skilled artisan will appreciate that certain factors may influence the dosage required to effectively treat a subject, including, but not limited to, the severity of the disease or disorder, previous treatments, the general health and/or age of the subject, and other diseases present. Moreover, treatment of a subject with a therapeutically effective amount of a composition can include a single treatment, or preferably, can include a series of treatments. The initial dose may be followed up with a booster dosage after a period of about 2 days to 2 weeks to maintain the innate immunity. Further booster dosages may also be administered.

The effective compositions of the present invention may be employed in such forms as capsules, liquid solutions, suspensions or elixirs for oral administration, or sterile liquid forms such as solutions or suspensions. Any inert acceptable carrier may preferably be used or any such acceptable carrier in which the compositions of the present invention have suitable solubility properties for use of the present invention.

Methods of administration will vary in accordance with the type of disorder and disease sought to be controlled or eradicated. The dosage of the composition will be dependent on a number of factors, including the route of administration. A person of ordinary skill in the art may easily and readily titrate the dosage for an enhanced immune response.

The actual effective amounts of compounds can vary according to the specific composition being utilized, the mode of administration, and the age, weight, and condition of the individual. As used herein, an effective amount of the drug is an amount which elicits or boosts an innate immune response. Dosages for a particular individual may be determined by a person of ordinary skill in the art using conventional considerations, e.g., by a means of appropriate, conventional pharmacological protocol.

The invention also provides kits for carrying out the therapeutic regimens of the invention. Such kits comprise in one or more containers therapeutically or prophylactically effective amounts of the compositions in a pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in the instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe preferably packaged in sterile form, for injecting the composition, and/or a packaged alcohol pad. Instructions are optionally included for administration of the composition by a clinician or by a patient.

Various cytokines, antibiotics, and other bioactive agents also may be coadministered with the compositions described herein. For example, various known cytokines, i.e., interleukin-1α(IL-1β), interleukin-1(IL-1β), interleukin-2 (IL-2), interleukin-3 (IL-3), interleukin-4 (IL-4), interleukin-5 (IL-5), interleukin-6 (IL-6), interleukin-7 (IL-7, interleukin-8 (IL-8), interleukin-9 (IL-9), interleukin-10 (IL-10), interleukin-11 (IL-11), IL-12, interferon-α(INFα), interferon-β(INFβ), interferon-γ(INFγ), tumor necrosis factor a, tumor necrosis factorβ(TNFβ), granulocyte colony stimulating factor (G-CSF), granulocyte/macrophage colony stimulating factor (GM-CSF), and transforming growth factorβ(TGF-β) may be co-administered with the composition in order to maximize the physiological response. However, it is anticipated that other but as yet undiscovered cytokines may be effective in the invention. In addition, conventional antibiotics may be co-administered wit the compositions. The choice of suitable antibiotics will, however, be dependent upon the disease in question.

The following examples are meant to be illustrative and not limiting in any way.

### EXAMPLES

A well-established animal model was used to assess whether different formulations of CpG oligodeoxynucleotide and QS-21 together or alone could function as stimulators of innate immunity. In brief, experiments were set up to compare QS-21 to a recently reported immunostimulatory CpG motif. An immunostimulatory CpG sequence (e.g., 1826), reported to serve as an adjuvant in mice, was selected. One experiment evaluated whether the CpG motif alone, QS-21 alone, or the CpG/QS-21 combination may serve to increase innate immunity by activation of natural killer cells.

The experiments were performed using materials from the following suppliers: QS-21 and QS-7 (Aquila Biopharmaceuticals); CpG oligodeoxynucleotides included the phosphorothiate-modified sequences 1826 TCCATGACGTTCCTGACGTT and 2006 TCGTCGTTTTGTCGTTTTGTCGTT (Life Technologies (Gibco)), murine recombinant IL-12 (Pharmingen), and YAC-1 cells (ATCC), a natural killer cell-sensitive target line.

### Example 1

### Natural Killer Cell Activity Induced by QS-21 and CpG/QS-21

Assessment of natural killer cell activity was carried out by an adaptation of a published method (Hashimoto et al., J. Immunol. 163: 583 (1999)). Balb/c mice (4 per group, female, 8-10 weeks of age) were administered one of five different candidate compositions at days 1 and 2. The compositions evaluated were (1) saline (negative control), (2) 10 ug QS-21, (3) 10 ug CpG (sequence 1826), (4) 0.5 ug murine IL-12 (positive control for NK cell activation), and (5) a combination of 10 ug QS-21 and 10 ug CpG in 0.2 ml saline. All test compositions were administered subcutaneously except for murine IL-12, which was administered intraperitoneally. Splenocytes were removed from the mice at day 3 for use as effector cells in the natural killer cell assay. Such cells were immediately used in a standard ⁵¹Cr release lysis assay. YAC-1 cells (loaded with ⁵¹Cr) were used as target cells. The lysis of this NK cell-sensitive line is indicative of NK cell activation in the splenocyte population.

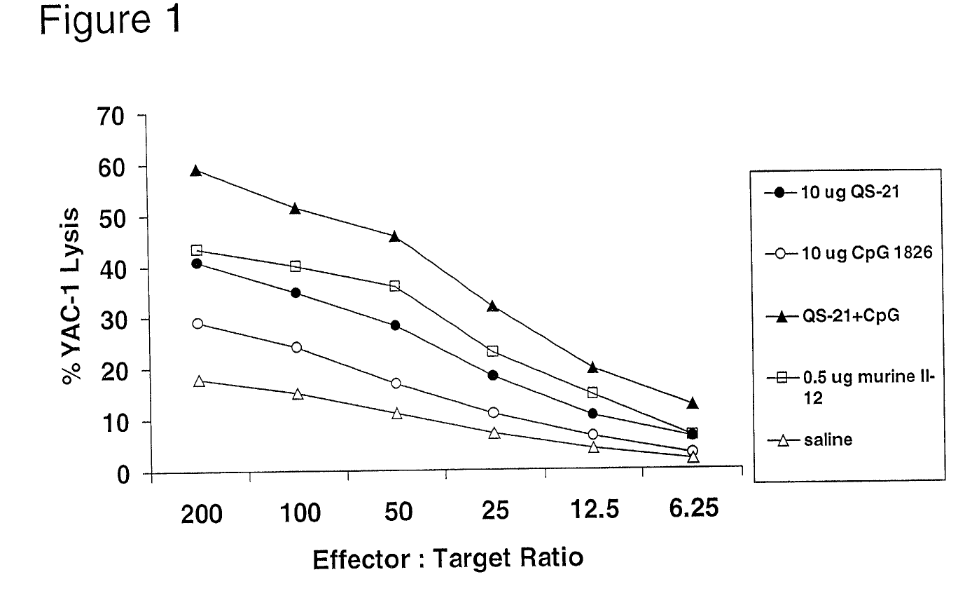

The results, as shown in the graphic representation of FIG. 1, indicate that minimal lysis (less than 20% at 100:1 effector to target ratio) was observed after the administration of saline. CpG alone slightly enhanced the NK cell activity. Surprisingly, QS-21 alone induced an NK cell response that was higher than CpG and that was nearly equivalent to the positive control, murine IL-12. Still more surprisingly, the combination of QS-21 and CpG induced the strongest NK cell response.

### Example 2

### Time Dependence of Natural Killer Cell

### Activity Induced by QS-21 and CpG/QS-21

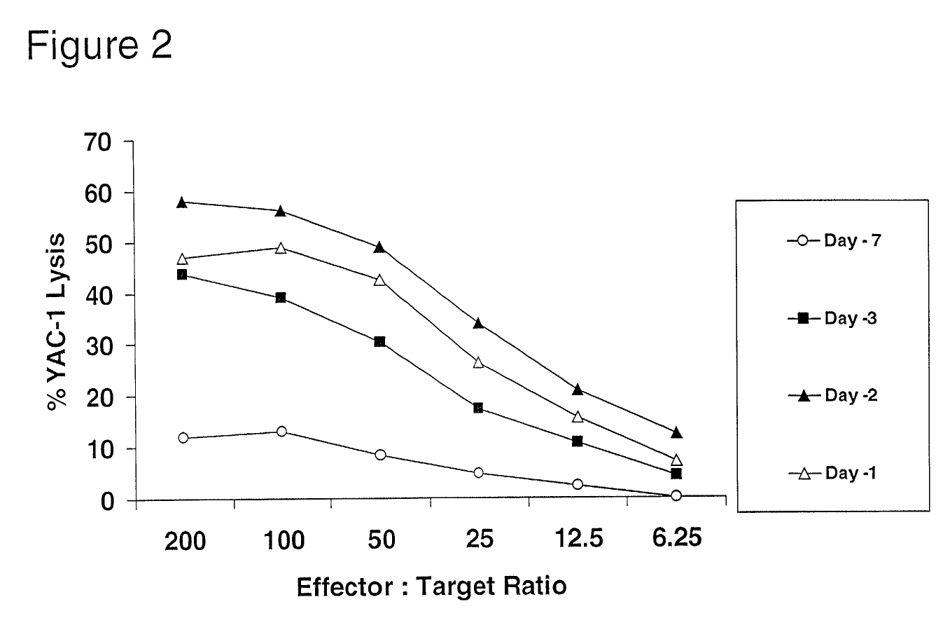

The time dependence of the administration of CpG/QS-21 on natural killer cell activation was investigated. Balb/c mice (5 per group, female, 8-10 weeks of age) were administered a mixture of 10 ug QS-21 and 10 ug CpG sequence 1826 in a total volume of 0.2 ml by subcutaneous route seven days before (−7d), three days before (−3d), two days before (−2d), and one day before (−1d) assay of natural killer cell activity. Splenocytes were removed from the mice at day 0 for use as effector cells in the natural killer cell assay. YAC-1 cells (loaded with ⁵¹Cr) were used as target cells. Natural killer cell lysis was apparent if the formulation of QS-21/CpG was administered one, two, or three days prior to the assay, but not if the formulation was administered seven days prior to the assay (FIG. 2). This confirms the transient nature of the natural killer cell activity.

### Example 3

### Dose Response of QS-21, QS-7, and CpG Sequence 1826

Balb/c mice (5 per group, female, 8-10 weeks of age) were administered individually QS-7, QS-21, or CpG sequence 1826 at three different dose levels (3, 10, 30 ug) to determine a dose response curve for these individual compounds. The compositions evaluated were (1) saline (negative control), (2) 3 ug QS-21, (3) 10 ug QS-21, (4) 30 ug QS-21, (5) 3 ug QS-7(6) 10 ug QS-7, (7) 30 ug QS-7, (8) 3 ug sequence CpG 1826, (9) 10 ug CpG sequence 1826, and (10) 30 ug CpG sequence 1826. All test compositions were administered subcutaneously at day 1 and day 2. Splenocytes were removed from the mice at day 3 for use as effector cells in the natural killer cell assay. YAC-1 cells (loaded with ⁵¹Cr) were used as target cells.

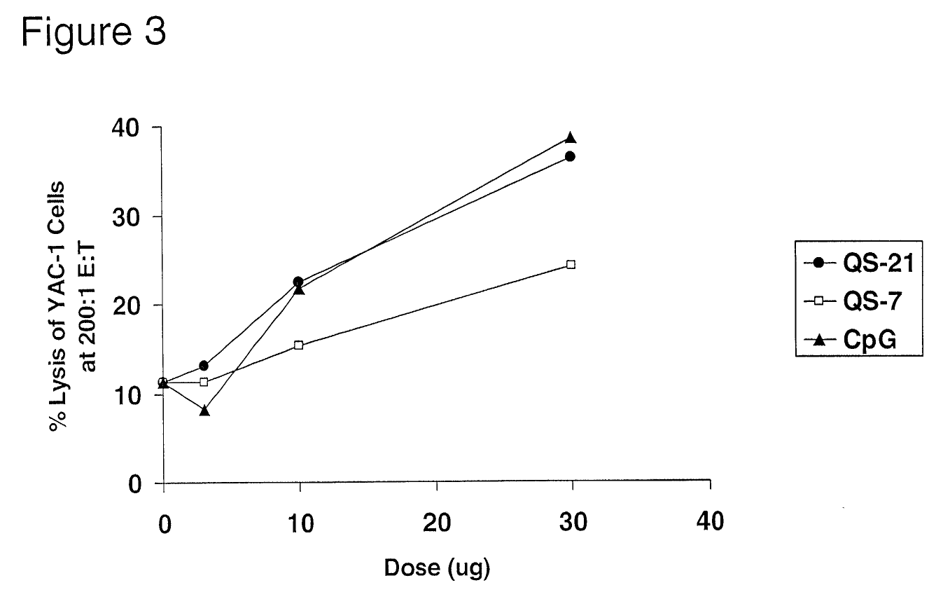

The results, as shown in the graphic representation of FIG. 3, confirm that QS-21, QS-7, and CpG sequence 1826 enhance NK activity in a dose dependent fashion. The NK cell activity induced by QS-21 or CpG sequence 1826 was higher than that induced by QS-7 at an equivalent dose. This experiment confirmed that NK activity could be induced by another saponin.

### Example 4

### NK Activity Induced by QS-21 and/or QS-7 and CpGs Sequences 1826 and 2006

This experiment evaluated the natural killer cell stimulating activity induced by various formulations: (1) QS-21 (10 ug), (2) QS-7 (10 ug), (3) CpG sequence 1826 (10 ug), (4) CpG sequence 2006 (10 ug), (5) QS-21 (10 ug)+CpG sequence 1826 (10 ug), (6) QS-21 (10 ug)+CpG 2006 (10 ug), (7) QS-7 (10 ug)+CpG sequence 1826 ( ug), (8) QS-7 (10 ug) +CpG sequence 2006 (10 ug), (9) QS-7 (10 ug) and QS-21 (10 ug) and (10) saline. Balb/c mice (5 per group, female, 8-10 weeks of age) were administered the above formulations by subcutaneous route on day 1 and day 2. Splenocytes were removed from the mice at day 3 for use as effector cells in the natural killer cell assay. YAC-1 cells (loaded with ⁵¹Cr) were used as target cells.

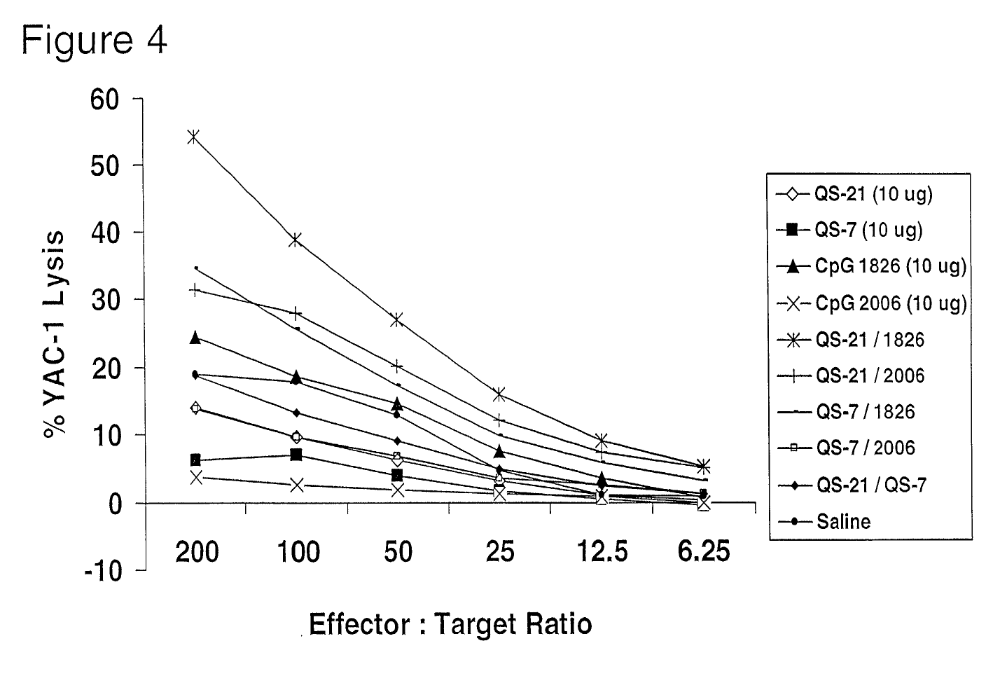

As evident in the graphic representation of FIG. 4, the results show that the three formulations inducing the strongest response are QS-21/CpG sequence 1826, QS-21/CpG sequence 2006, and QS-7/CpG sequence 1826. This indicates that mixtures of alternate CpG (sequence 2006) with QS-21 also lead to a heightened NK cell response; likewise mixtures of alternate saponins (QS-7) with CpG can also lead to a heightened NK cell response.

### Example 5

### Protection of Mice from Listeria Monocytogenes by

### Administration of Formulations that Enhance Innate Immunity

Another method of demonstration of enhanced innate immunity is in an in vivo challenge model. The protective benefit of formulations of QS-21 or QS-21/CpG was demonstrated in a Listeria monocytogenes challenge model in Balb/c mice. Immunity to Listeria monocytogenes can be mediated by innate immunity and is believed to rely on cytokines produced by natural killer cells (Harty, et al., Curr. Opin. Immunol 8:526) (1996)). Hence, this challenge model was used as a demonstration of the benefit of enhanced innate immunity raised by administration of the inventive compositions.

Balb/mice (5 per group, female, 8-10 weeks of age) were administered the following formulations on day 0: Group 1: saline, subcutaneous route. Group 2: 10 ug QS-21 and 10 ug of CpG sequence 1826, subcutaneous route. Group 3: 10 ug QS-21, subcutaneous route. Group 4: 10 ug CpG sequence 1826, subcutaneous route. Group 5: 0.5 ug recombinant murine IL-12, intraperitoneal route. A total volume of 0.2 ml was administered. On day 3, mice were challenged by the intraperitoneal route with 10⁵ colonies of Listeria monocytogenes strain 10403s. Spleens were removed at 96 hours after challenge, homogenized, and then cultured in serial 10-fold dilutions overnight on agar plates. Listeria monocytogenes colonies were counted, the number of organisms per spleen determined, and then the geometric mean and standard error were determined for each group. A two-tailed, paired student's t-test of the log10 colonies/spleen was used to show statistical significance.

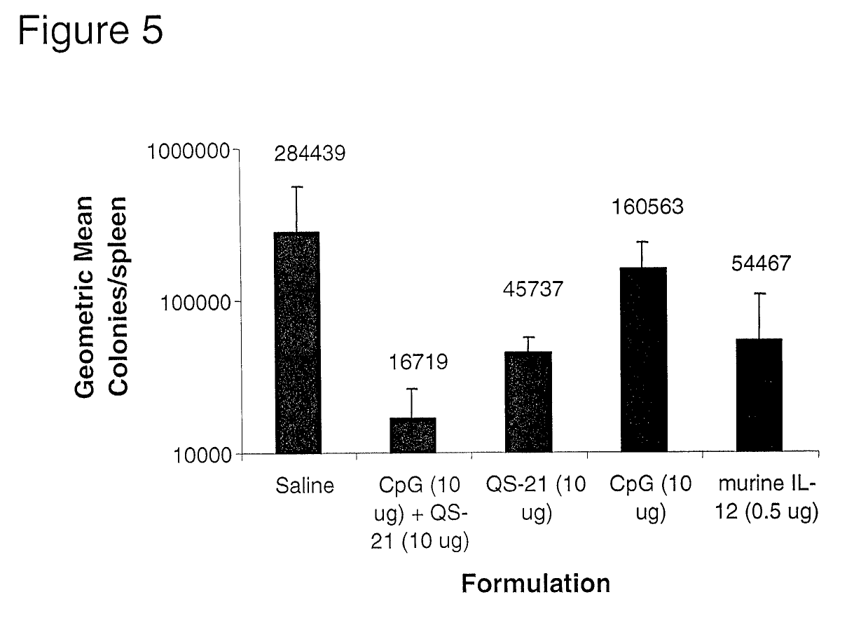

FIG. 5 is a graphic representation showing the results of the challenge. The group with the highest spleen colony count was the group receiving saline (control group). All other groups had lower mean colony counts in spleen. The lowest colony counts were in the CpG+QS-21 group and in the QS-21 group, both of which reached statistical significance (p<0.05). This suggests that these two formulations raise an innate immunity that is protective against a challenge with a bacterium.

The invention now being fully described, it will be apparent to one of ordinary skill in the art that many changes and modifications can be made thereto without departing from the spirit or scope of the invention as set forth below.

We claim:

1. A composition comprising:
(a) a saponin; and
(b) an oligonucleotide comprising at least one unmethylated CpG dinucleotide.

2. The composition as claimed in claim 1, wherein the saponin is derived from Quillaja saponaria.

3. The composition as claimed in claim 2, wherein the saponin is chemically modified.

4. The composition as claimed in claim 2, wherein the saponin comprises a substantially pure saponin.

5. The composition as claimed in claim 4, wherein the substantially pure saponin comprises QS-7, QS-17, QS-18, or QS-21.

6. The composition as claimed in claim 5, wherein the substantially pure saponin comprises QS-21.

7. The composition as claimed in claim 1, wherein the oligonucleotide is chemically modified.

8. The composition as claimed in claim 7, wherein the oligonucleotide is modified with at least one phosphorothioate internucleotide linkage.

9. The composition as claimed in claim 1, wherein the oligonucleotide comprises a CpG motif having the formula 5′X₁CGX₂3′, wherein at least one nucleotide separates consecutive CpGs, and wherein X₁ is adenine, guanine, or thymine, and X₂ is cytosine, thymine, or adenine.

10. The composition as claimed in claim 9, wherein the CpG motif comprises TCCATGACGTTCCTGACGTT or TCGTCGTTTTGTCGTTTTGTCGTT.

11. The composition as claimed in claim 1, wherein the composition increases an innate immune response when administered to a mammal.

12. The composition as claimed in claim 1, wherein the composition increases an innate immune response when administered to a human.

13. The composition as claimed in claim 1, wherein the composition increases an innate immune response when administered to a mammal other than a human.

14. The composition as claimed in claim 11, wherein the composition further enhances a natural killer cell response.

15. The composition as claimed in claim 14, wherein the composition further enhances a natural killer cell response in a positive synergistic manner.

16. A method for stimulating innate immunity comprising administering an effective amount of a composition comprising:
(a) a saponin; and
(b) an oligonucleotide comprising at least one unmethylated CpG motif to an individual.

17. The method as claimed in claim 16, wherein the saponin is derived from Quillaja saponaria.

18. The method as claimed in claim 16, wherein the saponin is chemically modified.

19. The method as claimed in claim 17, wherein the saponin comprises a substantially pure saponin.

20. The method as claimed in claim 19, wherein the substantially pure saponin comprises QS-7, QS-17, QS-18, or QS-21.

21. The method as claimed in claim 20, wherein the substantially pure saponin comprises QS-21.

22. The method as claimed in claim 16, wherein the oligonucleotide is chemically modified.

23. The method as claimed in claim 22, wherein the oligonucleotide is modified with at least one phosphorothioate internucleotide linkage.

24. The method as claimed in claim 16, wherein the oligonucleotide comprises a CpG motif having the formula 5′X₁,CGX₂3′, wherein at least one nucleotide separates consecutive CpGs, and wherein X₁ is adenine, guanine, or thymine, and X₂ is cytosine, thymine, or adenine.

25. The method as claimed in claim 24, wherein the CpG motif comprises TCCATGACGTTCCTGACGTT or TCGTCGTTTTGTCGTTTTGTCGTT.

26. The method as claimed in claim 16, wherein the composition stimulates an innate immune response when administered to a mammal.

27. The method as claimed in claim 16, wherein the composition stimulates an innate immune response when administered to a human.

28. The method as claimed in claim 16, wherein the composition stimulates an innate immune response when administered to a mammal other than a human.

29. The method as claimed in claim 16, wherein the method further enhances a natural killer cell response.

30. The method as claimed in claim 16, wherein the method further enhances a natural killer cell response in a positive synergistic manner.

31. A method for stimulating innate immunity comprising administering an effective amount of a composition comprising a saponin to an individual.

32. The method as claimed in claim 31, wherein the saponin is derived from Quillaja saponaria.

33. The method as claimed in claim 32, wherein the saponin is modified.

34. The method as claimed in claim 32, wherein the saponin comprises a substantially pure saponin.

35. The method as claimed in claim 34, wherein the substantially pure saponin comprises QS-7, QS-17, QS-18, or QS-21.

36. The method as claimed in claim 35, wherein the substantially pure saponin comprises QS-21.

37. The method as claimed in claim 32, wherein the composition stimulates an innate immune response when administered to a mammal.

38. The method as claimed in claim 32, wherein the composition stimulates an innate immune response when administered to a human.

39. The method as claimed in claim 32, wherein the composition stimulates an innate immune response when administered to a mammal other than a human.

40. The method as claimed in claim 32, wherein the method further enhances a natural killer cell response.

41. The method as claimed in claim 40, wherein the method further enhances a natural killer cell response in a positive synergistic manner.

42. The composition as claimed in claim 12, wherein the composition further enhances a natural killer cell response.

43. The composition as claimed in claim 13, wherein the composition further enhances a natural killer cell response.
